SEQUENCE LISTING (12 sequences)

SEQ ID NO: 1 - protein, 31 aa, Artificial Sequence
  Derived from human blood sample
ASPVAPLDPLLKYGRGQPVSSASGTTTDLGQ

SEQ ID NO: 2 - protein, 16 aa, Artificial Sequence
  Derived from human blood sample
KVGAHAGEYGAEALER

SEQ ID NO: 3 - protein, 18 aa, Artificial Sequence
  Derived from human blood sample
KTFTVTEGSMAGGAWGRL

SEQ ID NO: 4 - protein, 15 aa, Artificial Sequence
  Derived from human blood sample
KFLQAKFYQLSLERP

SEQ ID NO: 5 - protein, 20 aa, Artificial Sequence
  Derived from human blood sample
REVQGAFLRFMACLLKGYRV

SEQ ID NO: 6 - protein, 9 aa, Artificial Sequence
  Derived from human blood sample
RKEGKGERE

SEQ ID NO: 7 - protein, 18 aa, Artificial Sequence
  Derived from human blood sample
RMSIQCDVWRSKFLASRV

SEQ ID NO: 8 - protein, 16 aa, Artificial Sequence
  Derived from human blood sample
GVLLVRAPPACLQVLR

SEQ ID NO: 9 - protein, 16 aa, Artificial Sequence
  Derived from human blood sample
SSNNGCMRDPTLYRCK

SEQ ID NO: 10 - protein, 13 aa, Artificial Sequence
  Derived from human blood sample
GGVLSPADKTNVK

SEQ ID NO: 11 - protein, 18 aa, Artificial Sequence
  Derived from human blood sample
TLAGENQTALEIEELNRK

SEQ ID NO: 12 - protein, 14 aa, Artificial Sequence
  Derived from human blood sample
PVLTLDMPSSERGK

(19) United States
(12) Patent Application Publication
(10) Pub. No.: US 2010/0204128 A1
(43) Pub. Date: Aug. 12, 2010

(54) NOVEL PEPTIDES

(75) Inventors: Are Thoresen, Ramnes (NO); Sergio Manzetti, Nesoddtangen (NO)

Correspondence Address: VOLPE AND KOENIG, P.C., UNITED PLAZA, SUITE 1600, 30 SOUTH 17TH STREET, PHILADELPHIA, PA, 19103, US

(73) Assignee: SANARE INVESTMENT AS, Ramnes (NO)

(21) Appl. No.: 12/670,284

(22) PCT Filed: Jul. 10, 2008
(86) PCT No.: PCT/NO2008/000262

§ 371 Date: Jan. 22, 2010

(30) Foreign Application Priority Data
Jul. 24, 2007 (NO) 20073884

(51) Int. Cl. A61K 38/10; C07K 7/08; C07H 21/04; C07H 21/02; C12N 15/63; C12N 5/00; C12P 21/06; C07K 16/00; G01N 33/53

(52) U.S. Cl. 514/13; 530/326; 536/23.1; 435/320.1; 435/325; 435/69.1; 530/387.9; 435/7.1

(57) ABSTRACT

The present invention relates to novel peptides and mixtures thereof which have been shown anti-tumor activity. Further, the invention relates to methods for identifying such compounds as well as to methods for their production. DNA encoding said peptides, vectors, host organisms, pharmaceutical preparations and antibodies that specifically bind with said peptide are also a part of the present invention.

### FIELD OF THE INVENTION

The present invention relates to novel peptides and mixtures thereof which have been shown anti-tumor activity. Furthermore, the invention relates to methods for identifying such compounds as well as to methods for their production. DNA encoding said peptides, vectors, host organisms, pharmaceutical preparations and antibodies that specifically bind with said peptide are also a part of the present invention.

### BACKGROUND OF THE INVENTION

The major therapeutic approaches, with respect of the treatment of breast cancer, have evolved around endocrine therapies. Tamoxifen has been widely applied, but at a cost of several gynaecological and vasomotor symptoms². Other therapeutic approaches, such as non-specific aromatase inhibitors, have also been associated with adverse side effects. As a result of this, emerging third-generation aromatase inhibitors such as letrozole, anastrozole and exemestane have increasingly been applied². However, given their synthetic and foreign nature to the body's numerous mechanisms, their side-effects still proves to be “heavy-duty” for the patient's recovery and personal and psychosocial well-being. Thus there remains a need for improved therapeutic approaches of treating cancer, such as breast cancer, with less adverse side effects.

During the recent years, acupuncture has been increasingly used for treating long- and short-termed pathologies in human patients¹⁷. Acupuncture has been combined with chemotherapy in cancer treatment trials²⁵⁻³⁰, and has also been used for treating nausia caused by chemotherapy³²⁻³⁴. Furthermore, in 2003 it was reported that acupuncture may have a positive effect when treating breast cancer³⁸. However, the reason why acupuncture has a positive effect agains breast cancer cells has not previously been disclosed.

Based on a theory that acupuncture treatment may stimulate the formation/secretion of certain factors that inhibit proliferation of said breast cancer cells, the inventors of the present invention initiated a study that resulted in the identification of 12 peptides. Each of said 12 peptides were demonstrated to affect the proliferation of breast cancer cells, and a mixture of said 12 peptides was shown to inhibit the proliferation of the cancer cells. Said 12 peptides, hereinafter referred to as SEQIDNO1-12, have not previously been disclosed. However, sequences that are similar to SEQIDNO1, SEQIDNO2, SEQIDNO10 and SEQIDNO11 have been disclosed in the prior art.

EMBO J, 1987, 6(9):2767-2771 disclose a DNA sequence encoding a peptide that consists of 36 amino acids. Even though this peptide may be considered similar to SEQIDNO1 of the present invention, the prior art does not suggest a function of said peptide.

WO2003046556 relates to the identification of 3 peptides that may be used as disease markers. One of these peptides consists of 17 amino acids. This peptide differs from SEQIDNO2 in that it has a His residue in position 17. Although the two sequences are quite similar, the prior art does not disclose a function of the peptide, except that it may be used as a disease marker.

WO2005116607 relates to a method for the identification of Hb J-Toronto signature peptides. One of these signature peptides consists of 23 amino acids, wherein amino acid 1-11 in this peptide is identical to amino acid 3-13 in SEQIDNO10. In addition to the fact that SEQIDNO10 of the present invention is significantly shorter than the disclosed peptide, the prior art does not mention anything about the peptide's function.

WO2005114221 disclose hundreds of peptides that have been isolated from prostate cancer tissue. One of these peptides consists of 19 amino acids. This peptide differs from SEQIDNO11 in that it has two additional amino acids. Although the two sequences may be considered similar, the prior art does not suggest or mention a function of the peptide.

As mentioned above, there remains a need for improved therapeutic approaches of treating cancer, such as breast cancer. One such alternative approach is acupuncture treatment. In fact, acupuncture has been demonstrated to have a positive effect against breast cancer cells. However, the factors that are responsible for the positive effect of acupuncture treatment have not previously been disclosed.

One object of the present invention is to provide a method for the identification of the factors that are formed and/or secreted as a result of acupuncture treatment. It is also an object of the present invention to isolate or synthesise said factors. Said factors may then be used in the treatment of various disorders.

### SUMMARY OF THE INVENTION

A first aspect of the present invention relates to a peptide comprising an amino acid sequence having at least 80% sequence identity with an amino acid sequence selected from the group consisting of SEQIDNO5, SEQIDNO7, SEQIDNO8, SEQIDNO1 to SEQIDNO4, SEQIDNO6 and SEQIDNO9 to SEQIDNO12; or conservative modifications thereof.

A second aspect of the present invention relates to a nucleic acid encoding said peptide and a third and fourth aspect of the present invention relates to a vector comprising said nucleic acid and a suitable host organism comprising said nucleic acid and/or said vector respectively.

A fifth aspect of the present invention relates to a method of producing said peptide, comprising cultivating said host organism and isolating the peptide.

A sixth aspect of the present invention relates to a composition comprising a peptide according to the first aspect of the present invention.

Another aspect of the present invention relates to said peptide, said nucleic acid, said vector or said composition for medical use.

Further, the present invention relates to the use of said peptide, said nucleic acid, said vector or said composition for manufacturing a medicament for the treatment of cancer.

The present invention also relates to an antibody or antibody fragment that specifically binds with said peptide.

Furthermore, the present invention relates to a pharmaceutical formulation comprising said peptide, said nucleic acid, said vector and/or said composition; and a pharmaceutical acceptable vehicle.

The present invention also relates to a method of identifying potential drugs, comprising the following steps:
a) sampling of blood from a patient that suffers from a disease
b) stimulating a specific acupuncture point for a predetermined period of time
c) sampling of blood from the patient that has been subjected to acupuncture treatment
d) isolating a fraction of the blood sample obtained in step c) that has a protein/peptide content that is significantly different from the protein/peptide content in the corresponding fraction of the blood sample obtained in step a).
e) sequencing of the protein(s)/peptide(s) that is/are present in the fraction obtained in to step d).

Another aspect of the present invention relates to a method, preferably an in vitro method, of identifying potential drugs, comprising the following steps:
a) isolating the fraction of a blood sample A that has a protein/peptide content that is significantly different from the protein/peptide content in the corresponding fraction of blood sample B, wherein blood sample A has been sampled from a patient, prior to acupuncture treatment, that suffers from a disease and blood sample B has been sampled from the same patient subsequent to acupuncture treatment, wherein said acupuncture treatment involves stimulation of a specific acupuncture point for a predetermined period of time;
b) sequencing of the protein(s)/peptide(s) that is/are present in the fraction obtained in step a).

Preferred embodiments of the present invention are set forth in the dependent claims.

### DESCRIPTION OF THE FIGURES

FIG. 1

Shows the controlling sequences of the body processes according to Chinese acupuncture. In this system heart controls lung, lung controls liver, liver controls spleen/stomach, spleen/stomach controls kidney and kidney controls heart. If a disorder is related to the stomach, e.g. breast cancer^{38,39}, then the liver does not control the stomach properly³⁸. Consequently the liver has to be treated by stimulating an acupuncture point belonging to the liver meridian, e.g. LV03-T/LV03 (see FIG. 2).

FIG. 2

Shows the location of the LV03-T/LV03 (distal to point Liver 3 between Os metatarsale 1 and 2) acupuncture point on the human foot.

FIG. 3

First dimension analysis in the 2D-HPLC run (HPCF). The S0 line shows the protein content in the sample before acupuncture treatment, and the S1 line shows the protein content in the sample after acupuncture treatment.

FIG. 4

RP-HPLC plots for the second dimension, illustrating the signals (UV-absorbance) as intensity bands. The bands to the right illustrates the signals that were obtained from the S1 sample (after acupuncture treatment), and the bands to the left illustrates the signals that were obtained from the S0 sample (before acupuncture treatment). The central portion shows the signals as peaks of the RP-HPLC analysis.

FIG. 5

SEQ ID NO:1 on a T47D breast cancer cell line. Decreased fluorescent readings relatively to the DC5 sample indicates reduced cell growth.

DC5=Medium, Bap=Benzo[α]pyrene, DMSO=Dimethylsulfoxide.

FIG. 6

SEQ ID NO:2 on a T47D breast cancer cell line. Decreased fluorescent readings relatively to the DC5 sample indicates reduced cell growth.

DC5=Medium, Bap=Benzo[α]pyrene, DMSO=Dimethylsulfoxide.

FIG. 7

SEQ ID NO:3 on a T47D breast cancer cell line. Decreased fluorescent readings relatively to the DC5 sample indicates reduced cell growth.

DC5=Medium, Bap=Benzo[α]pyrene, DMSO=Dimethylsulfoxide.

FIG. 8

SEQ ID NO:4 on a T47D breast cancer cell line. Decreased fluorescent readings relatively to the DC5 sample indicates reduced cell growth.

DC5=Medium, Bap=Benzo[α]pyrene, DMSO=Dimethylsulfoxide.

FIG. 9

SEQ ID NO:5 on a T47D breast cancer cell line. Decreased fluorescent readings relatively to the DC5 sample indicates reduced cell growth.

DC5=Medium, Bap=Benzo[α]pyrene, DMSO=Dimethylsulfoxide.

FIG. 10

SEQ ID NO:6 on a T47D breast cancer cell line. Decreased fluorescent readings relatively to the DC5 sample indicates reduced cell growth.

DC5=Medium, Bap=Benzo[α]pyrene, DMSO=Dimethylsulfoxide.

FIG. 11

SEQ ID NO:7 on a T47D breast cancer cell line. Decreased fluorescent readings relatively to the DC5 sample indicates reduced cell growth.

DC5=Medium, Bap=Benzo[α]pyrene, DMSO=Dimethylsulfoxide.

FIG. 12

SEQ ID NO:8 on a T47D breast cancer cell line. Decreased fluorescent readings is relatively to the DC5 sample indicates reduced cell growth.

DC5=Medium, Bap=Benzo[α]pyrene, DMSO=Dimethylsulfoxide.

FIG. 13

SEQ ID NO:9 on a T47D breast cancer cell line. Decreased fluorescent readings relatively to the DC5 sample indicates reduced cell growth.

DC5=Medium, Bap=Benzo[α]pyrene, DMSO=Dimethylsulfoxide.

FIG. 14

SEQ ID NO:10 on a T47D breast cancer cell line. Decreased fluorescent readings relatively to the DC5 sample indicates reduced cell growth.

DC5=Medium, Bap=Benzo[α]pyrene, DMSO=Dimethylsulfoxide.

FIG. 15

SEQ ID NO:11 on a T47D breast cancer cell line. Decreased fluorescent readings relatively to the DC5 sample indicates reduced cell growth.

DC5=Medium, Bap=Benzo[α]pyrene, DMSO=Dimethylsulfoxide.

FIG. 16

SEQ ID NO:12 on a T47D breast cancer cell line. Decreased fluorescent readings relatively to the DC5 sample indicates reduced cell growth.

DC5=Medium, Bap=Benzo[α]pyrene, DMSO=Dimethylsulfoxide.

FIG. 17

Mixture of SEQ ID NO:1 through SEQ ID NO:12 on Tamoxifen resistant TMX2-28 cells. Decreased fluorescent readings relatively to the DC5 sample indicates reduced cell growth. The concentration of each of the 12 peptides in a specific sample is 1/12 of the concentration that is defined in the figure.

DC5=Medium, Bap=Benzo[α]pyrene, DMSO=Dimethylsulfoxide.

FIG. 18

Mixture of SEQ ID NO:1 through SEQ ID NO:12 on a MCF-7 breast cancer cell line. Decreased fluorescent readings relatively to the DC5 sample indicates reduced cell growth. The concentration of each of the 12 peptides in a specific sample is 1/12 of the concentration that is defined in the figure.

DC5=Medium, DMSO=Dimethylsulfoxide.

FIG. 19

Mixture of SEQ ID NO:1 through SEQ ID NO:12 on a T47D breast cancer cell line. Decreased fluorescent readings relatively to the DC5 sample indicates reduced cell growth. The concentration of each of the 12 peptides in a specific sample is 1/12 of the concentration that is defined in the figure.

DC5=Medium, Bap=Benzo[α]pyrene, DMSO=Dimethylsulfoxide.

### DETAILED DESCRIPTION OF THE INVENTION

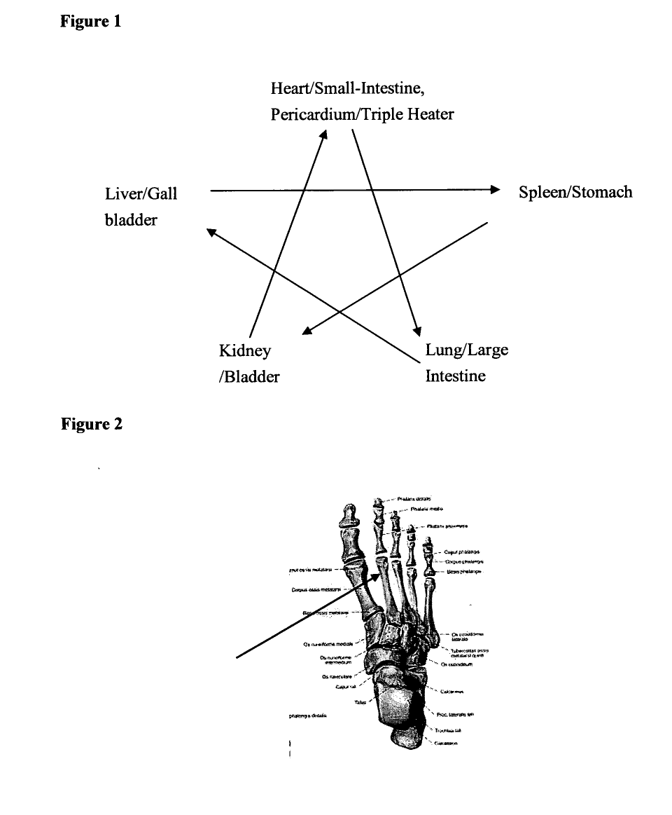

According to Thoresen, 2003³⁸, all the organs and organ processes may be involved in a sophisticated feed-back control system for cell-growth. This feed-back control system is described in Chinese acupuncture as the control-cycle. In this system, heart controls lung, lung controls liver, liver controls spleen/stomach, spleen/stomach controls kidney and kidney controls heart (FIG. 1). Accordingly, if a disease is related to the stomach, then the liver does not control the stomach properly³⁸. In order to treat said disease, an acupuncture point belonging to the liver median should be stimulated. Based on this theory, it might be possible that said acupuncture treatment stimulates the secretion of certain factors that may have a positive effect against said disease.

The present invention relates to a method of identifying potential drugs. Said method involves sampling of blood (S0) from a patient, e.g. from a patient suffering from breast cancer. Subsequently, a needle is applied to stimulate a specific acupuncture point for a predetermined period of time. In case the patient suffers from breast cancer, an acupuncture point belonging to the liver median (e.g. LV03-T/LV03) should be stimulated. Said predetermined period of time is preferably 1-60 minutes, even more preferably 1-30 minutes and most preferably 1-20 minutes. Subsequent to the acupuncture treatment, a blood sample is collected (S1).

The blood samples S0 and S1 are then fractionated in order to obtain a fraction of compounds that are filtered through a cut off filter, such as a 50 kDa, 40 kDa, 30 kDa, 20 kDa, 10 kDa or a 5 kDa cut off filter. Preferably, said cut off filter is a 10 kDa cut off filter. Said fractionation may e.g. be performed as explained in example 1.

Initial protein content estimates with the absorbance method (A₂₈₀-A₃₂₀) indicated a protein content of 0.48 mg/ml in the 10 kDa S0 fraction (before stimulus) and a protein content of 1.02 mg/ml in the 10 kDa S1 fraction (after stimulus). The increase in protein content indicates that a significant effect was mediated by the acupuncture treatment.

To further study the effect of acupuncture treatment, the two fractions (10 kDa S0 and 10 kDa S1) were independently subjected to a method that is suitable to identify the part of the fraction that is responsible for the observed change in protein content. The use of 2D-HPLC, as explained in example 1, is one example of such a suitable method. Said change in protein content may be an increase or a decrease, such as an increase.

The parts of the 10 kDa S1 fraction that was demonstrated a higher or lower protein content compared with the corresponding part of the 10 kDa S0 fraction were independently subjected to a method that is suitable for peptide identification. The use of electrospray mass spectral analysis, as explained in example 1, is one example of such a suitable method.

Preferably, the parts of the 10 kDa S1 fraction that was demonstrated a 10% increase/decrease in protein content relatively to the corresponding part of the 10 kDa S0 fraction were independently subjected to a method that is suitable for peptide identification. Even more preferably, the parts of the 10 kDa S1 fraction that was demonstrated a 20% increase/decrease in protein content relatively to the corresponding part of the 10 kDa S0 fraction were independently subjected to a method that is suitable for peptide identification.

A 10% or 20% increase/decrease in protein content should be understood as a 10% or 20% increase/decrease in the signal intensity obtained by the method described in example 1 (sample analysis).

The sequence of each identified peptide was then estimated, e.g. by using BioWorks SeQuest Analysis Software package (Shevchenko and Chernushevich, 1997), as explained in example 1. Based on the estimated peptide sequences, twelve different peptides were synthesized.

A first aspect of the present invention relates to a peptide comprising an amino acid sequence having at least 60% sequence identity with an amino acid sequence selected from the group consisting of SEQIDNO1 to SEQIDNO12, preferably SEQIDNO5, SEQIDNO7 or SEQIDNO8; or conservative modifications thereof. Preferably said sequence identity is at least 70%, more preferably at least 80% and even more preferably at least 90%, such as 100%. Preferably, all of said derivatives and variants of SEQIDNO1 have a biological activity that is similar to the peptide represented by SEQIDNO1. The same applies to SEQIDNO2-12.

One of skill will recognize that individual substitutions, deletions or additions to a peptide, polypeptide, or protein sequence which alters, adds or deletes a single amino acid or a small percentage of amino acids in the encoded sequence is a “conservative modification” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Histidine (H), Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

In one embodiment according to the first aspect of the present invention, said peptide consists of an amino acid sequence having at least 60% sequence identity with an amino acid sequence selected from the group consisting of SEQIDNO1 to SEQIDNO12, preferably SEQIDNO5, SEQIDNO7 or SEQIDNO8; or conservative modifications thereof. Preferably said sequence identity is at least 70%, more preferably at least 80% and even more preferably at least 90%, such as 100%. Preferably, all of said derivatives and variants of SEQIDNO1 have a biological activity that is similar to the peptide represented by SEQIDNO1. The same applies to SEQIDNO2-12.

In one embodiment according to the second aspect of the present invention, said amino acid sequence is selected from the group consisting of:

### SEQIDNO2 to SEQIDNO12;

### SEQIDNO1 and SEQIDNO3-12;

### SEQIDNO1 to SEQIDNO9 and SEQIDNO11-12;

### SEQIDNO1 to SEQIDNO10 and SEQIDNO12;

or conservative modifications thereof.

In another embodiment according to the first aspect of the present invention, said peptides do not comprise an amino acid sequence selected from the group consisting of:

MTPFASPVAPLDPLLKYGRGQGPVSSASGTTTDLG;
KVGAHAGEYG AEALERH;
VLSPADKTNVKAAWGKVGAHAGE;
and
RTLAGENQTAFEIEELNRK.

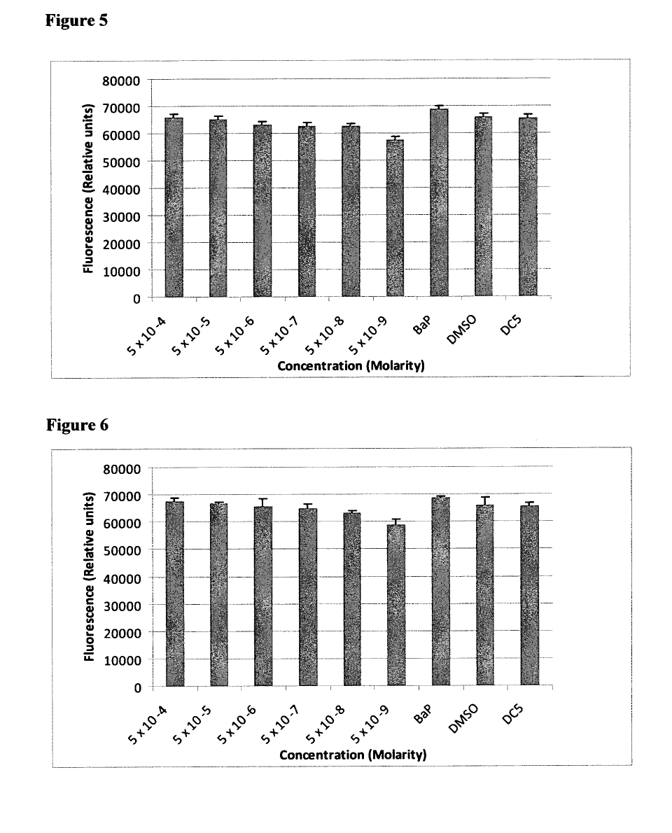

While some of the peptides according to the first aspect of the present invention have been shown to inhibit proliferation of TMX-2-28, MCF-7 and T47D cells, others induced growth and yet others apparently had no significant effect (FIG. 5-16 and table 1-12). However, a linear dose-response curve was observed after 24 hours of incubation with a mixture of said peptides, which indicates a strong growth inhibitory effect (FIG. 16-19). Accordingly, the peptides according to the first aspect of the present invention may be useful individually or in mixtures.

A sixth aspect of the present invention relates to a composition comprising a peptide according to the first aspect of the present invention. Preferably, said composition comprises at least two of said peptides, and even more preferably one of the at least two peptides is the peptide represented by SEQIDNO5, SEQIDNO7 or SEQIDNO8. Most preferably said composition comprises all of the three last-mentioned peptides. One example of such a composition is a composition comprising each and all of the twelve peptides according to the first aspect of the present invention.

In one embodiment according to the sixth aspect of the present invention, said composition comprises the peptides represented by SEQIDNO2 to SEQIDNO7, SEQIDNO10 and SEQIDNO11. In another embodiment, said composition comprises the peptides represented by SEQIDNO1 to SEQIDNO7, SEQIDNO10 and SEQIDNO11. In yet another embodiment, said composition comprises the peptides represented by SEQIDNO2 to SEQIDNO8, SEQIDNO10 and SEQIDNO11.

A second aspect of the present invention relates to a nucleic acid molecule encoding the peptide according to the first aspect of the present invention; or conservative modifications thereof. Said nucleic acid may be DNA or RNA. The nucleic acid sequence can be deduced by the skilled artisan on the basis of the disclosed amino acid sequences.

With respect to the nucleic acid sequences, conservative modifications refers to those nucleic acids which encode identical or essentially identical amino acid sequences, or where the nucleic acid does not encode an amino acid sequence, to essentially identical is or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

A third aspect of the present invention relates to a vector comprising the nucleic acid according to the second aspect of the present invention. The vector can be of any type suitable e.g. for expression of said peptides or propagation of genes encoding said peptides in a particular organism. The specific choice of vector depends on the host organism and is known to a person skilled in the art.

A fourth aspect of the present invention relates to a suitable host organism comprising the nucleic acid according to the second aspect of the present invention, and/or the vector according to the third aspect of the present invention. The host organism may be of eukaryotic or prokaryotic origin.

A fifth aspect of the present invention relates to a method of producing the peptide according to the first aspect of the present invention comprising cultivating the host organism according to the fourth aspect of the present invention and isolating the peptide.

An seventh aspect of the present invention relates to the peptide according to the first aspect of the present invention, the composition according to the sixth aspect of the present invention, the nucleic acid according to the second aspect of the present invention or the vector according to the third aspect of the present invention for medical use.

A eighth aspect of the present invention relates to the use of the peptide according to the first aspect of the present invention, the composition according to the sixth aspect of the present invention, the nucleic acid according to the second aspect of the present invention or the vector according to the third aspect of the present invention for manufacturing a medicament for the treatment of cancer. Preferably said cancer is breast cancer or intestinal cancer (e.g. colon cancer).

A ninth aspect of the present invention relates to an antibody or an antibody fragment that specifically binds with the peptide according to the first aspect of the present invention.

A tenth aspect of the present invention relates to a pharmaceutical formulation comprising the peptide according to the first aspect of the present invention, the composition according to the sixth aspect of the present invention, the nucleic acid according to the second aspect of the present invention or the vector according to the third aspect of the present invention.

Having now fully described the present invention in some detail by way of illustration and example for purpose of clarity of understanding, it will be obvious to one of ordinary skill in the art that same can be performed by modifying or changing the invention by with a wide and equivalent range of conditions, formulations and other parameters thereof, and that such modifications or changes are intended to be encompassed within the scope of the appended claims.

### EXAMPLES

The following examples are meant to illustrate how to make and use the invention. They are not intended to limit the scope of the invention in any manner or to any degree.

### Example 1

### Identification of 12 Peptides, Hereinafter Refereed to as SEQIDNO1-12

### Acupuncture and Sample Collection

A sample (S0) of 7 ml blood was collected from a female patient aged 43 with breast cancer with spreading to the bones. The sample was collected in a solution of Guanidinium Chloride to yield a final concentration of 6M Guanidinium Chloride. The acupuncture needle, which was sterilized and ideally of gold or stainless steel, was inserted in the designated point LV03-T/LV03 (FIG. 2). The second sample, S1, was taken 1 minute after insertion of the needle and mixed with Guanidinium Chloride.

### Sample Fractionation

Samples were then fractionated in 5 steps at 3000 rpm in a Eppendorff Centrifuge, first with a spin-column filter of 0.45 μm to remove larger intracellular components, second with a 0.20 μm spin-column filter to remove cellular components, third at a 300 kDa cut-off to remove large proteins and eventual cellular bodies, then 100 kDa to remove larger and small proteins and at last 10 kDa cut off to obtain an oligopeptide fraction.

### Sample Analysis

100 μL of the 10 kDa fractions of the S0 and S1 samples were analysed with respect to the total amount of protein, using the absorbance method (A₂₈₀-A₃₂₀). The result of this analysis indicates that the protein content in the 10 kDa fraction of the S0-sample (before stimulus) was around 0.48 mg/ml and that the protein content in the 10 kDa fraction of S1-sample (after stimulus) was around 1.02 mg/ml. The increase in protein content indicates that a significant effect was mediated by the acupuncture treatment.

### 2D-HPLC Analysis

The 10 kDa fractions of the S0 and S1 samples were then subjected to 2D liquid chromatography with a Beckman's Proteome Lab™ PF 2D system. The samples were cleared at 100,000 g centrifugation and exchanged with start buffer (6M urea, 25 mM bis-tris, 0.2% n-octyl-β-D-glucopyranoside, pH 8.5 with ammonium hydroxide) on a PD10 column. The samples were then subjected to separation on a HPCF (High Performance ChromatoFocusing) column pre-equilibrated with the “start buffer”. Samples were monitored for UV absorbance (280 nm) and pH in a flow cell during the entire run. 1 mg of sample was injected with a flow rate of 0.2 ml/minute. After the initial 35 min start buffer load, an isocratic gradient was generated by switching to the eluent buffer (6M urea, 10% v/v Polybuffer™ 74 (GE Healthcare), 0.2% n-octyl-β-D-glucopyranoside, pH4 with iminodiacetic acid) for 95 min. Fraction collection occurred every 5 min or if a pH change of 0.3 was recorded. Upon completion of the pH gradient, a 20 minute 1M NaCl was run during which fractions were collected for 5 min intervals.

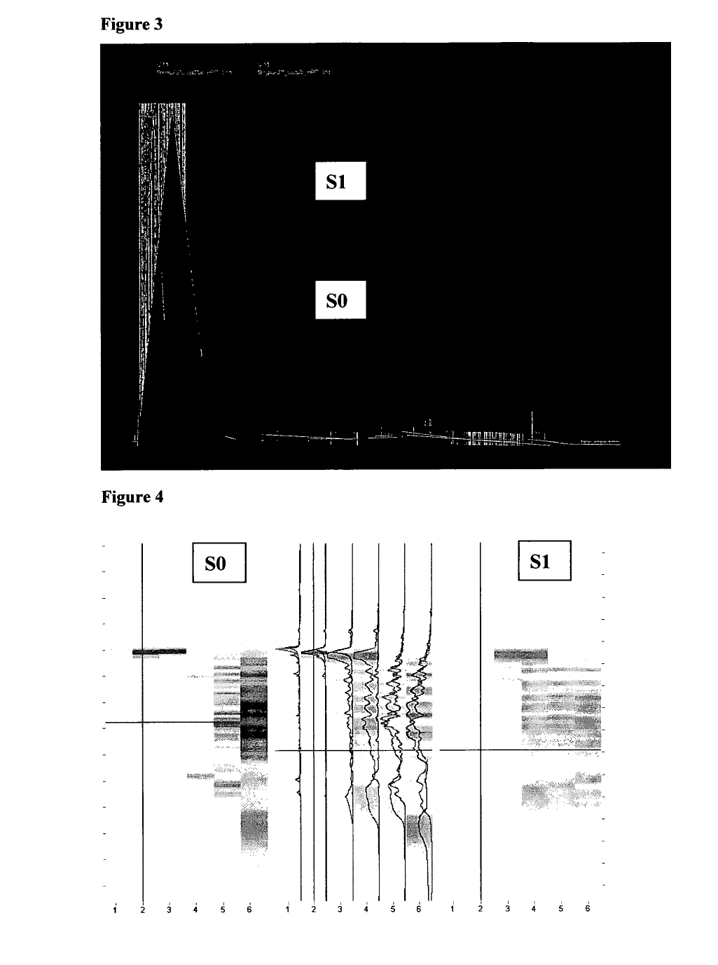

The run in the first dimension (IEC) revealed significant differences between the S0 and S1 samples, which were largely observed in the run-period ranging between 0-35 min (see FIG. 3). The fractions identified in the 0-35 min time-interval from the first dimension was therefore subjected to to separation by HPRP (High Performance Reverse Phase).

A HPRP column was equilibrated with 0.1% TFA, and 200 ul of the selected HPCF fractions were injected at a flow rate of 0.750 ml/mn. Fractions were monitored for UV absorbance at 214 nm. Immediately after injection, an 0-100% acetonitrile/0.8% TFA gradient was run for 30 min. HPRP fractions were then collected on a Gilson fraction collector set at 0.25-0.5 minute per fraction. UV absorbance data from samples were imported into the ProteoVue™ software package for a visual display of the UV absorbance. Comparison between two samples will be done by importing separate ProteoVue™ analysis representing two different sample into the DeltaVue™ software package. This allows direct comparison of the UV absorbance profile of two samples analyzed under identical run parameters. The run in the second dimension revealed significant differences between the S0 and S1 samples, as shown in FIG. 4, lane 2, 3 and 4.

Proteins were subjected to in-solution tryptic digestion (Schevchenko and Schevchenko, 2003), on the Genomic Solution™ (Ann Arbor, Mich.) ProPrep™ robot in the Department of Biochemistry, Molecular Biology and Biophysics' Proteomic Analysis Core facility, University of Minnesota. Samples were reduced in the presence of 10 mM DTT/25 mM NH₄HCO₃ at 60° for 30 minutes followed with the addition of iodacetamide (55 mM final concentration)/25 mM NH₄HCO₃ and incubated for 30 minutes at 25°. Tryptic digestion in the presence of 12 ng/μl trypsin (Promega, Madison, Wis.) in 25 mM NH₄HCO₃, 5 mM CaCl₂ at 37° was preformed for 10 hours. Formic acid was added to a final concentration of 0.1% w/v to stop the digestion. The sample was then frozen and concentrated in a speed vacuum.

### Electrospray Mass Spectral Analysis

Samples were rehydrated in loading buffer (30 μl of 98:2 water:ACN, 0.1% trifluoroacetic acid) and loaded onto a Michrom C-18 nanotrap by sample aspiration of 27.5 μl into a 100 μl sample loop using load buffer as the transfer reagent on a Michrom BioResources Paradigm AS1. The column was switched in-line with a capillary column allowing peptides elution at 350 nl/min with the Michrom BioResourceMS4. The capillary column (75 μm internal diameter) was packed in-house to 12 cm length with 5 m, 200 Å pore size C¹⁸ particles (Michrom BioResources, Auburn, Calif.) as described in (Mosely et al., 1997). Peptides were eluted with a linear gradient with 100% solvent A (95:5 water:ACN, 0.1% formic acid), to a final solvent B (5:95 water:ACN, 0.1% formic acid). The LC system was online with ThermoFinnigan. (ABI, Inc., Foster City, Calif.) LTQ ion trap mass spectrometer (MS). An electrospray spray voltage of 2250 V was applied distal to the analytical column. The instrument's calibration is monitored using the [M+2H]²⁺ average peak at 811 m/z (Sigma-Aldrich, Inc., St. Louis, Mo.). As peptides eluted from the column they were focused into the mass spectrometer where product ion spectra were collected in a data dependent acquisition (DDA) mode.

### Sequence Analysis

Mass spectra were analyzed using BioWorks SeQuest Analysis Software package. (Shevchenko and Chernushevich, 1997) Product ion mass spectra were searched using BioWorks (ABI, Inc., Foster City, Calif.) against a human database (Apr. 22, 2004) for protein identification. The following search parameters were used: 2 trypsin missed cleave site; peptide and product ion tolerance=1.0 Dalton; variable amino acids incorporated into the search were carbamidomethyl cysteine, singly oxidized methionine; and deamidation of glutamine and asparagine. 12 peptides were identified, hereinafter referred to as SEQIDNO1 to SEQIDNO 12.

### Peptide Synthesis

Peptides SEQ ID NO:1 to SEQ ID NO:12 were synthesized using conventional peptide synthesis equipment.

### Example 2

### Anti Tumor Activity

Stock solutions for each of the twelve synthesised peptides were prepared in basic culture medium (DMEM) at a concentration of 0.01 M. Several of the compounds did not go 100% into solution at this concentration and continued effort was directed towards appropriately diluting the peptides using 0.1% dimethylsulfoxide (DMSO) for the highest peptide concentrations.

Re-feed media were prepared to yield the following final six concentrations of each of the 12 peptides: 5×10⁻³, 5×10⁻⁴, 5×10⁻⁵, 5×10⁻⁶, 5×10⁻⁷, and 5×10⁻⁸M. The mixtures were then prepared containing equal volumes of peptides SEQ ID NO:1 through SEQ ID NO:12 (equal volumes of said re-feed media) to yield the following final six concentrations: 5×10⁻³, 5×10⁻⁴, 5×10⁻⁵, 5×10⁻⁶, 5×10⁻⁷, and 5×10⁻⁸M. With that, the 5×10⁻³M mixture contains about 0.42×10⁻³M of each peptide. Furthermore, mixtures containing equal volumes of peptides SEQ ID NO:2-SEQ ID NO:7, SEQ ID NO:10 and SEQ ID NO:11 were also prepared (equal volumes of said re-feed media) to yield the following final six concentrations: 5×10⁻³, 5×10⁻⁴, 5×10⁻⁵, 5×10⁻⁶, 5×10⁻⁷, and 5×10⁻⁸ M. With that, the 5×10⁻³M mixture contains about 0.42×10⁻³M of each of said 8 peptides.

MCF-7 and T47D cells were seeded into 96-well plates at a density of 10,000 cells per well in 90 μL of appropriate culture medium. TMX2-28 cells were seeded into 96-well plates at a density of 5,000 cells per well in 90 μL of appropriate culture medium. Plates were placed in a 37° C., 5% CO₂ incubator and cells were allowed to attach to the bottom of the well for 24 hours after which 10 μL of the appropriate refeed medium was added. Each concentration was tested in duplicate in each cell line. In addition, DC5, DMSO and either tamoxifen or BaP were present on each plate. Cells were then incubated for 24 hours. Subsequently 10 μL of AlmarBlue (Biosource) was added to each well. After three hours, the fluorescence was detected using a Packard Instrument Plate Reader with 535/20 excitation and 590/20 emission filters.

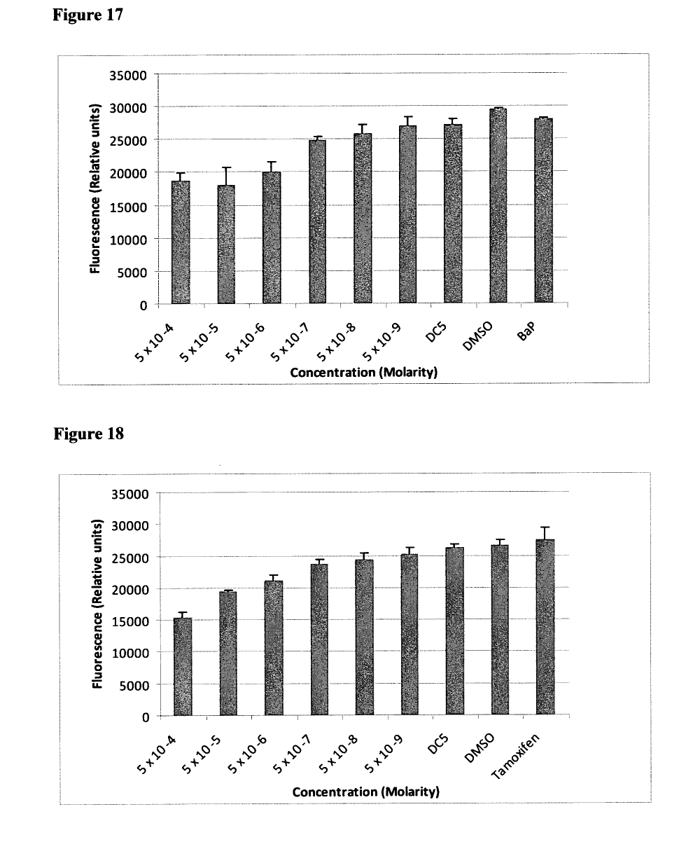

As shown in FIG. 5-16 and table 1-12, some of the peptides of the present invention inhibited proliferation of TMX-2-28, MCF-7 and T47D cells, others induced growth and yet others apparently had no significant effect. Further, as shown in FIG. 17-19 and table 13, a mixture of the 12 peptides was shown to inhibit the proliferation of the cells in a dose-dependent manner. Furthermore, a mixture of SEQIDNO2-7+SEQIDNO10-11 was also found to inhibit cell growth (table 14). These results are particularly interesting in light of the fact that the incubation with the peptide mixture was for 24 hours only, a time-period that was insufficient to decrease the proliferation by either Tamoxifen or Benzo[α]pyrene.

### Tables

TABLE 1
SEQIDNO1 activity studies.
TMX2-28 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 74129 | 73649 | 72975 | 74503 | 75445 | 71409 | 52745 | 70158 | 72202 |
| 72743 | 71774 | 71640 | 75697 | 77164 | 71588 | 59998 | 73977 | 69445 |

MCF-7 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | Tamoxifen | DMSO | DC5 |
| 69131 | 65474 | 65860 | 62613 | 63693 | 62279 | 62147 | 62484 | 60560 |
| 67176 | 66009 | 64140 | 62686 | 62463 | 60719 | 61921 | 65064 | 54197 |

T47D cells (illustrated in FIG. 5)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 65814 | 64794 | 63241 | 63036 | 62633 | 55767 | 68709 | 67910 | 66572 |
| 66151 | 65757 | 63091 | 62709 | 62689 | 59593 | 69129 | 63991 | 64694 |

Decreased fluorescent readings relatively to the DC5 sample indicates reduced cell growth. Bap (1 × 10⁻⁶ M) and tamoxifen (5 × 10⁻⁶ M) are positive controls.
DC5 = Medium, Bap = Benzo[a]pyrene, DMSO = Dimethylsulfoxide.

TABLE 2
SEQIDNO2 activity studies.
TMX2-28 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 70121 | 70162 | 69083 | 72557 | 72061 | 68910 | 52745 | 70158 | 72202 |
| 70743 | 68517 | 71861 | 73134 | 73603 | 72372 | 59998 | 73977 | 69445 |

MCF-7 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | Tamoxifen | DMSO | DC5 |
| 68630 | 66492 | 65617 | 63699 | 63539 | 63298 | 62147 | 62484 | 60560 |
| 65876 | 66554 | 65531 | 63627 | 63477 | 62132 | 61921 | 65064 | 54197 |

T47D cells (illustrated in FIG. 6)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 66780 | 67257 | 63525 | 64064 | 62463 | 57744 | 68709 | 67910 | 66572 |
| 68474 | 66607 | 67453 | 66021 | 63876 | 60399 | 69129 | 63991 | 64694 |

Decreased fluorescent readings relatively to the DC5 sample indicates reduced cell growth. Bap (1 × 10⁻⁶ M) and tamoxifen (5 × 10⁻⁶ M) are positive controls.
DC5 = Medium, Bap = Benzo[a]pyrene, DMSO = Dimethylsulfoxide.

TABLE 3
SEQIDNO3 activity studies.
TMX2-28 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 68963 | 69237 | 71007 | 72558 | 72686 | 69066 | 52745 | 70158 | 72202 |
| 67344 | 66454 | 67496 | 69648 | 69197 | 70821 | 59998 | 73977 | 69445 |

MCF-7 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | Tamoxifen | DMSO | DC5 |
| 63831 | 64053 | 64217 | 62452 | 61563 | 62603 | 62147 | 62484 | 60560 |
| 65178 | 64928 | 64899 | 66233 | 63132 | 61621 | 61921 | 65064 | 54197 |

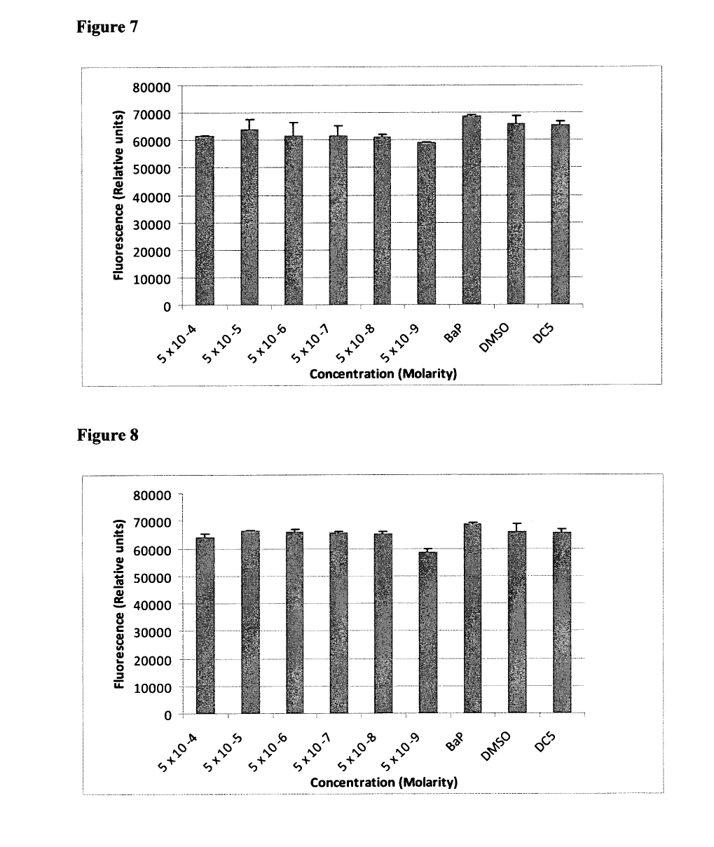

T47D cells (illustrated in FIG. 7)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 61359 | 66736 | 65079 | 64329 | 61766 | 59482 | 68709 | 67910 | 66572 |
| 61718 | 61724 | 58554 | 59141 | 61031 | 59451 | 69129 | 63991 | 64694 |

Decreased fluorescent readings relatively to the DC5 sample indicates reduced cell growth. Bap (1 × 10⁻⁶ M) and tamoxifen (5 × 10⁻⁶ M) are positive controls.
DC5 = Medium, Bap = Benzo[a]pyrene, DMSO = Dimethylsulfoxide.

TABLE 4
SEQIDNO4 activity studies.
TMX2-28 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 68299 | 67939 | 69917 | 70744 | 71761 | 70255 | 52745 | 70158 | 72202 |
| 67310 | 68552 | 70618 | 72469 | 72585 | 70823 | 59998 | 73977 | 69445 |

MCF-7 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | Tamoxifen | DMSO | DC5 |
| 64475 | 63637 | 65699 | 64278 | 64878 | 61356 | 62147 | 62484 | 60560 |
| 66716 | 66597 | 66767 | 64885 | 64702 | 61062 | 61921 | 65064 | 54197 |

T47D cells (illustrated in FIG. 8)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 65000 | 66449 | 66808 | 65859 | 66040 | 57800 | 68709 | 67910 | 66572 |
| 63427 | 66303 | 65677 | 65314 | 64619 | 59394 | 69129 | 63991 | 64694 |

Decreased fluorescent readings relatively to the DC5 sample indicates reduced cell growth. Bap (1 × 10⁻⁶ M) and tamoxifen (5 × 10⁻⁶ M) are positive controls.
DC5 = Medium, Bap = Benzo[a]pyrene, DMSO = Dimethylsulfoxide.

TABLE 5
SEQIDNO5 activity studies.
TMX2-28 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 27056 | 62564 | 67405 | 69438 | 70141 | 69376 | 60075 | 71189 | 72712 |
| 29836 | 67140 | 69619 | 69605 | 70591 | 67302 | 61350 | 71192 | 67410 |

MCF-7 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | Tamoxifen | DMSO | DC5 |
| 56679 | 61089 | 61618 | 61677 | 62738 | 57537 | 62999 | 71558 | 65433 |
| 54497 | 62458 | 65571 | 66372 | 64281 | 60120 | 67975 | 68854 | 65607 |

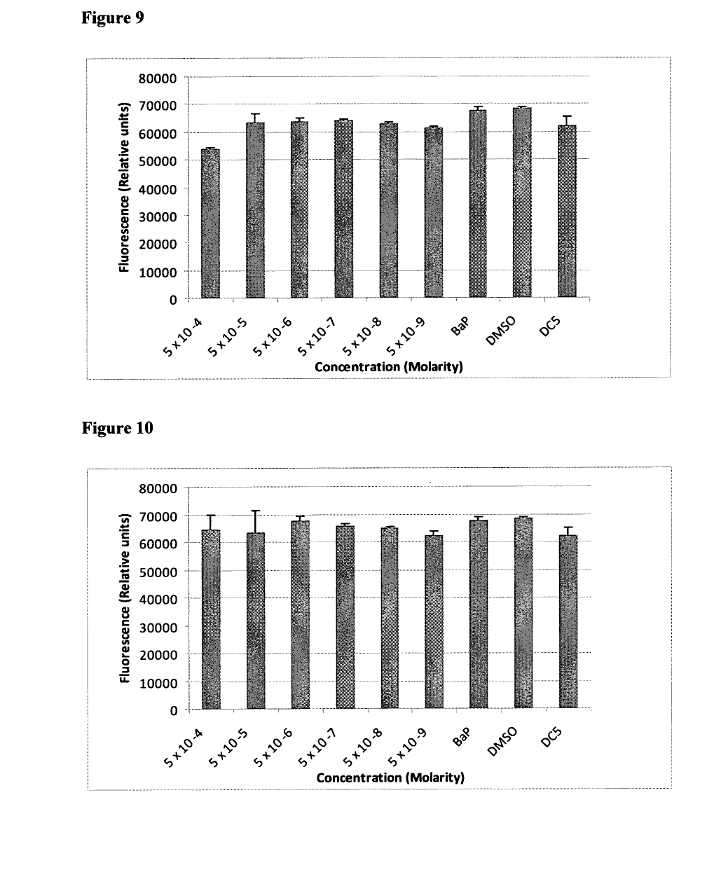

T47D cells (illustrated in FIG. 9)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 54287 | 61500 | 63089 | 64043 | 62339 | 61350 | 67072 | 68276 | 64397 |
| 54068 | 65566 | 64735 | 64408 | 63402 | 61820 | 68671 | 68910 | 60369 |

Decreased fluorescent readings relatively to the DC5 sample indicates reduced cell growth. Bap (1 × 10⁻⁶ M) and tamoxifen (5 × 10⁻⁶ M) are positive controls.
DC5 = Medium, Bap = Benzo[a]pyrene, DMSO = Dimethylsulfoxide.

TABLE 6
SEQIDNO6 activity studies.
TMX2-28 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 68949 | 70355 | 70389 | 71096 | 70707 | 62308 | 60075 | 71189 | 72712 |
| 68566 | 69358 | 69887 | 70512 | 71208 | 64367 | 61350 | 71192 | 67410 |

MCF-7 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | Tamoxifen | DMSO | DC5 |
| 69282 | 69071 | 67961 | 66967 | 65221 | 60855 | 62999 | 71558 | 65433 |
| 66723 | 69114 | 70454 | 68329 | 65987 | 61454 | 67975 | 68854 | 65607 |

T47D cells T47D cells (illustrated in FIG. 10)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 60992 | 57868 | 66902 | 65508 | 65244 | 61392 | 67072 | 68276 | 64397 |
| 68394 | 68983 | 68862 | 66620 | 64730 | 63577 | 68671 | 68910 | 60369 |

Decreased fluorescent readings relatively to the DC5 sample indicates reduced cell growth. Bap (1 × 10⁻⁶ M) and tamoxifen (5 × 10⁻⁶ M) are 5 positive controls.
DC5 = Medium, Bap = Benzo[a]pyrene, DMSO = Dimethylsulfoxide.

TABLE 7
SEQIDNO7 activity studies.
TMX2-28 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 58887 | 64931 | 68010 | 68325 | 67109 | 62010 | 60075 | 71189 | 72712 |
| 60090 | 66366 | 67591 | 70586 | 70471 | 66328 | 61350 | 71192 | 67410 |

MCF-7 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | Tamoxifen | DMSO | DC5 |
| 64171 | 67257 | 66311 | 63995 | 65755 | 60589 | 62999 | 71558 | 65433 |
| 62704 | 64433 | 66105 | 67019 | 66292 | 62763 | 67975 | 68854 | 65607 |

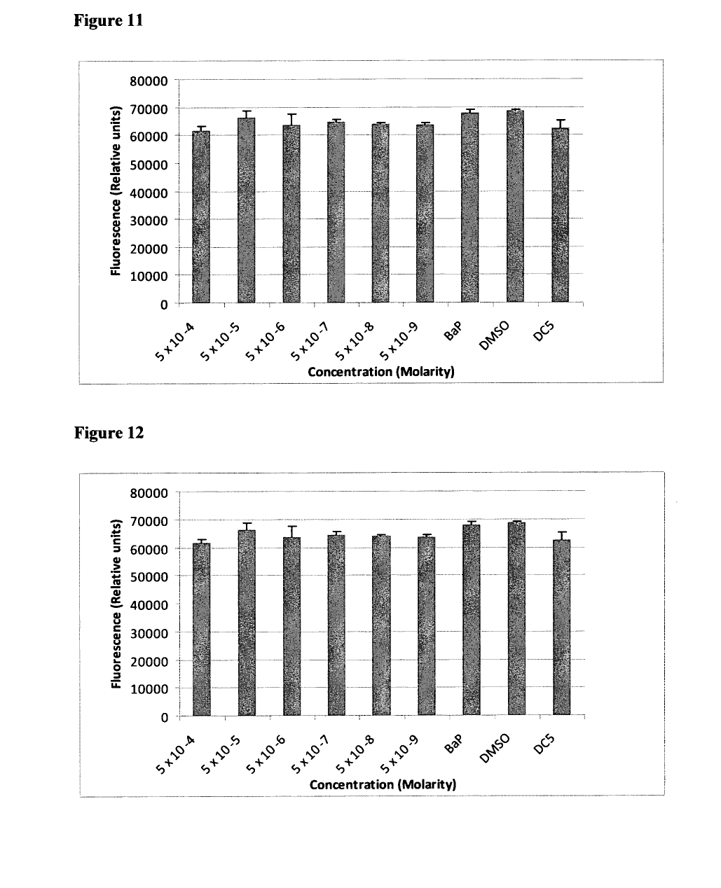

T47D cells (illustrated in FIG. 11)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 62688 | 68041 | 66479 | 65388 | 64353 | 64124 | 67072 | 68276 | 64397 |
| 60767 | 64777 | 60908 | 63864 | 63685 | 63134 | 68671 | 68910 | 60369 |

Decreased fluorescent readings relatively to the DC5 sample indicates reduced cell growth. Bap (1 × 10⁻⁶ M) and tamoxifen (5 × 10⁻⁶ M) are positive controls.
DC5 = Medium, Bap = Benzo[a]pyrene, DMSO = Dimethylsulfoxide.

TABLE 8
SEQIDNO8 activity studies.
TMX2-28 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 63114 | 67475 | 68833 | 70559 | 70603 | 68676 | 60075 | 71189 | 72712 |
| 65457 | 69324 | 70414 | 72074 | 73593 | 68864 | 61350 | 71192 | 67410 |

MCF-7 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | Tamoxifen | DMSO | DC5 |
| 64002 | 68720 | 69897 | 68985 | 68423 | 61653 | 62999 | 71558 | 65433 |
| 64485 | 70447 | 70376 | 70404 | 68263 | 66015 | 67975 | 68854 | 65607 |

T47D cells (illustrated in FIG. 12)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 62688 | 68041 | 66479 | 65388 | 64353 | 64124 | 67072 | 68276 | 64397 |
| 60767 | 64777 | 60908 | 63864 | 63685 | 63134 | 68671 | 68910 | 60369 |

Decreased fluorescent readings relatively to the DC5 sample indicates reduced cell growth. Bap (1 × 10⁻⁶ M) and tamoxifen (5 × 10⁻⁶ M) are positive controls.
DC5 = Medium, Bap = Benzo[a]pyrene, DMSO = Dimethylsulfoxide.

TABLE 9
SEQIDNO9 activity studies.
TMX2-28 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 71608 | 69255 | 69234 | 67663 | 67229 | 68009 | 56076 | 67625 | 65584 |
| 70176 | 69328 | 68280 | 69155 | 67807 | 65131 | 57832 | 68347 | 62965 |

MCF-7 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | Tamoxifen | DMSO | DC5 |
| 67879 | 65563 | 65046 | 63071 | 57728 | 62556 | 63504 | 64678 | 63670 |
| 68176 | 69218 | 65931 | 65962 | 62231 | 61065 | 65399 | 63807 | 61809 |

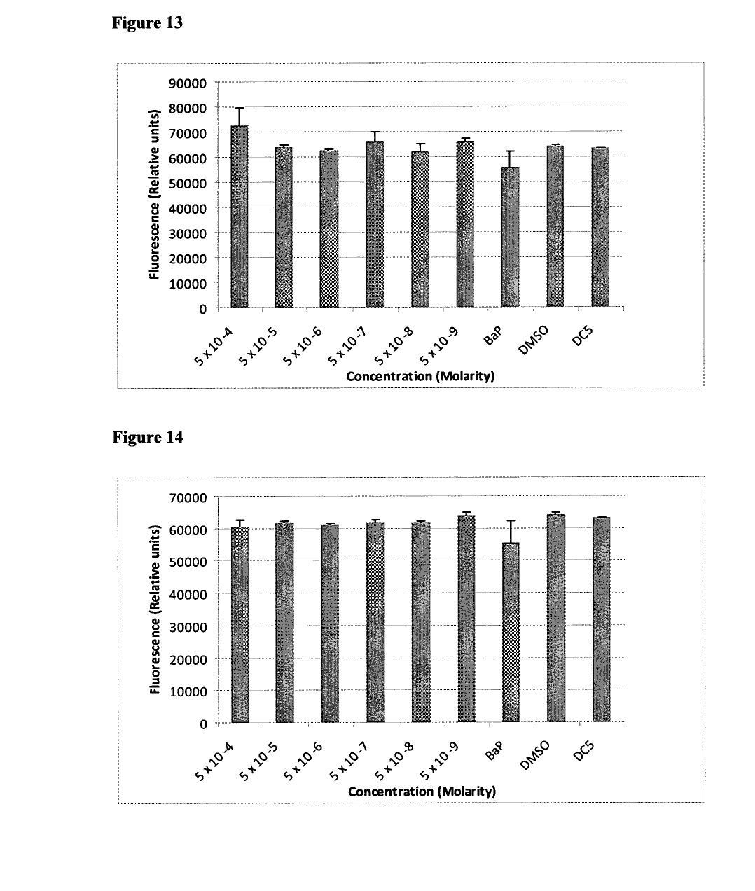

T47D cells (illustrated in FIG. 13)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 67492 | 63181 | 62848 | 63130 | 60146 | 65094 | 50994 | 64632 | 63285 |
| 77427 | 64535 | 62359 | 68689 | 64467 | 67070 | 60368 | 63641 | 63372 |

Decreased fluorescent readings relatively to the DC5 sample indicates reduced cell growth. Bap (1 × 10⁻⁶ M) and tamoxifen (5 × 10⁻⁶ M) are positive controls.
DC5 = Medium, Bap = Benzo[a]pyrene, DMSO = Dimethylsulfoxide.

TABLE 10
SEQIDNO10 activity studies.
TMX2-28 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 74950 | 70143 | 68926 | 68674 | 67904 | 64358 | 56076 | 67625 | 65584 |
| 67668 | 70096 | 71006 | 69541 | 70174 | 65678 | 57832 | 68347 | 62965 |

MCF-7 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | Tamoxifen | DMSO | DC5 |
| 66888 | 67041 | 68442 | 67343 | 62238 | 62410 | 63504 | 64678 | 63670 |
| 71581 | 68134 | 69458 | 65315 | 64184 | 63264 | 65399 | 63807 | 61809 |

T47D cells (illustrated in FIG. 14)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 58905 | 62235 | 61232 | 61576 | 62058 | 63213 | 50994 | 64632 | 63285 |
| 62040 | 61799 | 61422 | 62488 | 61865 | 64672 | 60368 | 63641 | 63372 |

Decreased fluorescent readings relatively to the DC5 sample indicates reduced cell growth. Bap (1 × 10⁻⁶ M) and tamoxifen (5 × 10⁻⁶ M) are positive controls.
DC5 = Medium, Bap = Benzo[a]pyrene, DMSO = Dimethylsulfoxide.

TABLE 11
SEQIDNO11 activity studies.
TMX2-28 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 66600 | 67045 | 66568 | 64371 | 65587 | 64433 | 56076 | 67625 | 65584 |
| 67446 | 66528 | 64759 | 65373 | 66707 | 65703 | 57832 | 68347 | 62965 |

MCF-7 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | Tamoxifen | DMSO | DC5 |
| 65114 | 65947 | 64058 | 63837 | 60430 | 60886 | 63504 | 64678 | 63670 |
| 72373 | 68983 | 64030 | 64861 | 65118 | 63795 | 65399 | 63807 | 61809 |

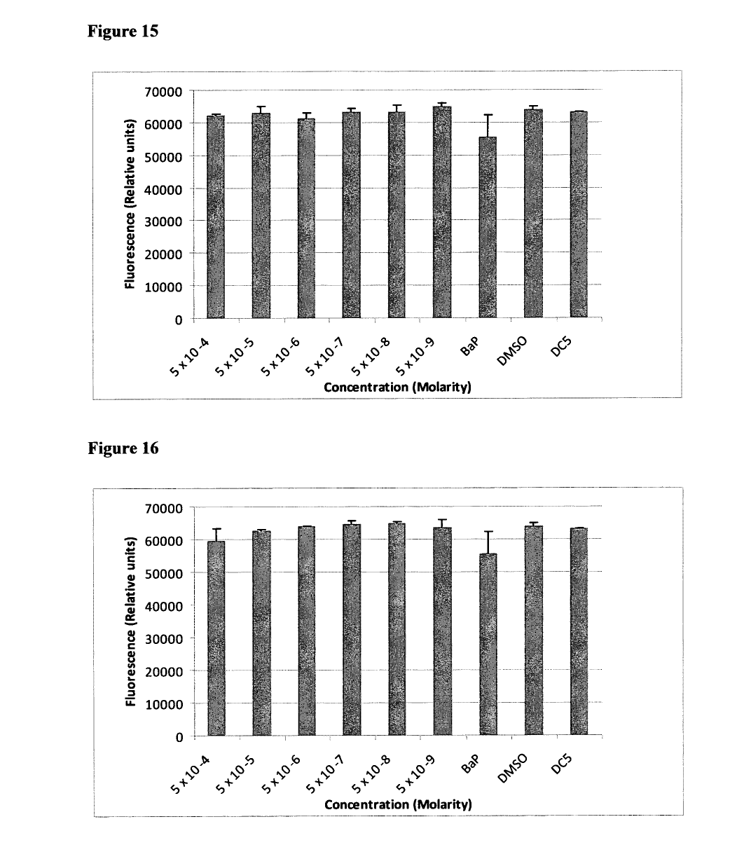

T47D cells (illustrated in FIG. 15)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 61976 | 61546 | 62448 | 62557 | 62018 | 63935 | 50994 | 64632 | 63285 |
| 62453 | 64267 | 60200 | 63900 | 64649 | 65696 | 60368 | 63641 | 63372 |

Decreased fluorescent readings relatively to the DC5 sample indicates reduced cell growth. Bap (1 × 10⁻⁶ M) and tamoxifen (5 × 10⁻⁶ M) are positive controls.
DC5 = Medium, Bap = Benzo[a]pyrene, DMSO = Dimethylsulfoxide.

TABLE 12
SEQIDNO12 activity studies.
TMX2-28 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 64801 | 67335 | 69868 | 67062 | 68382 | 66458 | 56076 | 67625 | 65584 |
| 66313 | 67800 | 69586 | 68845 | 67167 | 66191 | 57832 | 68347 | 62965 |

MCF-7 cells

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | Tamoxifen | DMSO | DC5 |
| 64634 | 67452 | 67604 | 65967 | 64530 | 59810 | 63504 | 64678 | 63670 |
| 66223 | 68292 | 67923 | 68650 | 64993 | 61717 | 65399 | 63807 | 61809 |

T47D cells (illustrated in FIG. 16)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | BaP | DMSO | DC5 |
| 57184 | 62230 | 64065 | 64023 | 65019 | 62179 | 50994 | 64632 | 63285 |
| 62215 | 62786 | 63826 | 65295 | 65289 | 65227 | 60368 | 63641 | 63372 |

Decreased fluorescent readings relatively to the DC5 sample indicates reduced cell growth. Bap (1 × 10⁻⁶ M) and tamoxifen (5 × 10⁻⁶ M) are positive controls.
DC5 = Medium, Bap = Benzo[a]pyrene, DMSO = Dimethylsulfoxide.

TABLE 13
Activity studies on a mixture comprising SEQIDNO1-12.
TMX2-28 (illustrated in FIG. 17)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | DC5 | DMSO | BaP |
| 19618 | 19938 | 19149 | 24420 | 26842 | 27997 | 26593 | 29683 | 28167 |
| 17966 | 16195 | 21134 | 25207 | 25027 | 25967 | 27751 | 29462 | 27829 |

MCF-7 (illustrated in FIG. 18)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | DC5 | DMSO | Tamoxifen |
| 15917 | 19169 | 20614 | 23282 | 23677 | 25949 | 26653 | 27209 | 28761 |
| 14811 | 19624 | 21839 | 24219 | 25099 | 24607 | 25764 | 26113 | 26110 |

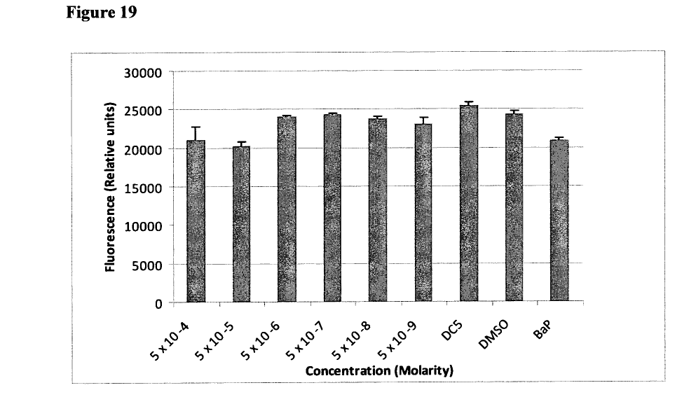

T47D (illustrated in FIG. 19)

| Concentration(Molarity) | | | | | | Controls | | |
|---|---|---|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ | DC5 | DMSO | BaP |
| 19911 | 19942 | 24175 | 24407 | 23984 | 23633 | 25127 | 24113 | 20732 |
| 22269 | 20661 | 23915 | 24233 | 23571 | 22438 | 25805 | 24706 | 21197 |

Decreased fluorescent readings relatively to the DC5 sample indicates reduced cell growth. Bap (1 × 10⁻⁶ M) and tamoxifen (5 × 10⁻⁶ M) are positive controls.
DC5 = Medium, Bap = Benzo[a]pyrene, DMSO = Dimethylsulfoxide.

TABLE 14
Activity studies on a mixture
comprising SEQIDNO2-7 + SEQIDNO10-11.
Concentration(Molarity)

| | | | | | |
|---|---|---|---|---|---|
| 5 × 10⁻⁴ | 5 × 10⁻⁵ | 5 × 10⁻⁶ | 5 × 10⁻⁷ | 5 × 10⁻⁸ | 5 × 10⁻⁹ |

TMX2-28 cells

| | | | | | |
|---|---|---|---|---|---|
| 18046 | 18061 | 24875 | 25179 | 27474 | 28777 |
| 16175 | 24487 | 27451 | 25866 | 26799 | 28223 |

MCF-7 cells

| | | | | | |
|---|---|---|---|---|---|
| 16309 | 19277 | 22516 | 23270 | 24537 | 24714 |
| 20269 | 23225 | 23895 | 23205 | 24024 | 25069 |

T47D cells

| | | | | | |
|---|---|---|---|---|---|
| 19565 | 22096 | 22449 | 23776 | 23486 | 24580 |
| 21973 | 22865 | 23128 | 23092 | 23427 | 24804 |

Decreased fluorescent readings indicates reduced cell growth.

### REFERENCES

2. Coster S, and Fallowield L. J. (2002). The impact of endocrine therapy on patients with breast cancer: a review of the literature. The Breast, 11, 1-12
17. Wong R, Sagar C M, Sagar S M. (2001). Abstract Integration of Chinese medicine into supportive cancer care: a modern role for an ancient tradition. Cancer Treat Rev. 27: 235-46.
25. Song L C, Liu C Y, Zhang B P, Wang T, Song Y Q, Li Y W. (1994). Electrochemical therapy (ECT) for thyroid adenoma during acupuncture anaesthesia: analysis of 46 patients. Eur. J. Surg. Suppl. 574:79-81.
26. Crocetti E, Crotti N, Feltrin A, Ponton P, Geddes M, Buiatti E. U. O. (1998). The use of complementary therapies by breast cancer patients attending conventional treatment. Eur. J. Cancer 34:324-8
27. Jacobson J S, Workman S B, Kronenberg F. (2000). Research on complementary/alternative medicine for patients with breast cancer: a review of the biomedical literature. J. Clin. Oncol. 18:668-83
28. Boon H, Stewart M, Kennard M A, Gray R, Sawka C, Brown J B, McWilliam C, Gavin A, Baron R A, Aaron D, Haines-Kamka T. (2000). Use of complementary/alternative medicine by breast cancer survivors in Ontario: prevalence and perceptions. J. Clin. Oncol. 18:2515-21
29. Shen J, Glaspy J. (2001). Acupuncture: evidence and implications for cancer supportive care. Cancer Pract. 9:147-50
30. Tagliaferri M, Cohen I, Tripathy D. (2001). Complementary and alternative medicine in early-stage breast cancer. Semin. Oncol. 28:121-34.
32. Dundee J W, Chestnutt W N, Ghaly R G, Lynas A G. (1986). Traditional Chinese acupuncture: a potentially useful antiemetic? Brit. Med. J. 293:583-4.
33. Dundee J W, Ghaly R G, Bill K M, Chestnutt W N, Fitzpatrick K T, Lynas A G. (1989). Effect of stimulation of the P6 antiemetic point on postoperative nausea and vomiting. Brit. J. Anaesth. 63:612-8.
34. Aglietti L, Roila F, Tonato M, Basurto C, Bracarda S, Picciafuoco M, Ballatori E, Del Favero A. (1990). A pilot study of metoclopramide, dexamethasone, diphenhydramine and acupuncture in women treated with cisplatin. Cancer Chemother. Pharmacol. 26:239-40
38. Thoresen A. (2003). Klinische Zwischenergebnisse meiner Studien über Akupunktur bei der Krebsbehandlung: Notizen aus meiner Sammlung von Fallbeispielen. Zeitschrift für Ganzheitliche Tiermedizin 17: 159-163.© MVS Medizinverlage Stuttgart GmbH & Co. KG
39. Ross J (1995). Acupuncture Point Combinations: the Key to Clinical Success. 1st Edition, Churchill Livingstone ISBN-10: 0443050066

1. A peptide comprising an amino acid sequence having at least 80% sequence identity with an amino acid sequence selected from the group consisting of SEQIDNO5, SEQIDNO7 and SEQIDNO8, said peptide having anti-proliferative activity against TMX2-28.

2. The peptide according to claim 1, wherein the amino acid sequence selected from said group is SEQIDNO5.

3. The peptide according to claim 1, wherein the amino acid sequence selected from said group is SEQIDNO7.

4. The peptide according to claim 1, wherein the amino acid sequence selected from said group is SEQIDNO8.

5. The peptide according to claim 1, said peptide consisting of an amino acid sequence having at least 80% sequence identity with an amino acid sequence selected from the group consisting of SEQIDNO5, SEQIDNO7 and SEQIDNO8.

6. The peptide according to claim 1, wherein said sequence identity is 100%.

7-9. (canceled)

10. A nucleic acid molecule encoding the peptide according to claim 1.

11. The nucleic acid molecule according to claim 10, wherein said nucleic acid molecule is DNA or RNA.

12. A vector comprising the nucleic acid molecule according to claim 10.

13. A suitable host organism comprising the nucleic acid molecule according to claim 10.

14. A method of producing the peptide according to claim 1, comprising cultivating the host organism of claim 13 and isolating the peptide.

15-22. (canceled)

23. An antibody or a fragment thereof that specifically binds with the peptide according to claim 1.

24. A pharmaceutical formulation comprising the peptide according to claim 1; and a pharmaceutical acceptable vehicle.

25. (canceled)

26. A method of identifying potential drugs comprising the following steps:
a) isolating the fraction of a blood sample A that has a protein/peptide content that is significantly different from the protein/peptide content in the corresponding fraction of blood sample B, wherein blood sample A has been sampled from a patient, prior to acupuncture treatment, that suffers from a disease and blood sample B has been sampled from the same patient subsequent to acupuncture treatment, wherein said acupuncture treatment involves stimulation of a specific acupuncture point for a predetermined period of time;
b) sequencing of the protein(s)/peptide(s) that is/are present in the fraction obtained in step a).

27. The method of claim 26, wherein said disease is breast cancer and said acupuncture point is LV03-T/LV03.

28. The method according to claim 26, wherein said predetermined period of time is 1-20 minutes.

29. The method according to claim 26, wherein a significantly different protein/peptide content represents a protein content difference of ±10%.

30. The peptide according to claim 1, said peptide having anti-proliferative activity against one or more of MCF-7 cells and T47D cells.

31. The peptide according to claim 1, wherein said peptide is able to pass a 10 kDa cut off filter at 3000 rpm.

32. A method of treating cancer comprising administering one or more selected from the group consisting of the peptide according to claim 1, the nucleic acid according to claim 10, the vector according to claim 12, and the composition according to claim 15.

33. A pharmaceutical formulation comprising a pharmaceutically acceptable vehicle and one or more selected from the group consisting of the peptide according to claim 1, the nucleic acid according to claim 10, the vector of claim 12, and the composition according to claim 15.
